(19) United States
(12) Patent Application Publication
(10) Pub. No.: US 2007/0173895 A1
(43) Pub. Date: Jul. 26, 2007

(54) Device for cardiac stimulation

(75) Inventor: Klaus Reichenbach, Glottertal (DE)

Correspondence Address: Orum & Roth LLC;Ste 1616, 53 W. Jackson Blvd., Chicago, IL, 60604, US

(73) Assignee: Iivetec Ingenieurburo GmbH

(21) Appl. No.: 11/598,883

(22) Filed: Nov. 14, 2006

(30) Foreign Application Priority Data
Nov. 15, 2005 (DE) 102005054783.4

(51) Int. Cl. A61N 1/362

(52) U.S. Cl. 607/10

(57) ABSTRACT

A cardiac stimulation device is proposed, which is equipped[U1] with leads (2, 12) for at least two stimulating electrodes, a combined input and display device (3, 13) for entering a mode of operation and the associated parameters suitable for the treatment as well as for displaying the selected mode of operation and parameters. A processor is provided for defining electrical stimulating pulses from the selected mode of operation and parameters. A stimulation device connected to the leads (2, 12) generates the electrical stimulating pulses defined by the processor.

The invention involves a cardiac stimulation device according to the preamble of claim 1.

Cardiac stimulation devices can be implanted in the body or worn outside the body. The device delivers electrical stimulating pulses to the heart via stimulation electrodes that end in the atrium or ventricle, which in turn reacts with rhythmic contractions. Such devices are also known as cardiac pacemakers. In many cases the heart is monitored by sensors, which are also located in the electrodes. The cardiac stimulation device adapts the delivery of pulses to the heart's needs. In a special mode of operation, stimulating pulses are delivered to the heart only if the heart's natural rhythm is too slow.

During cardiac arrhythmias, changes of the heartbeat sequence are caused by electrical stimuli or conduction disorders. These are usually triggered by underlying heart conditions such as coronary insufficiency, myocardial infarction and carditis as well as non-cardiac pathologies such as electrolyte disorders or hormonal diseases. Consequences can include an abnormally fast heartbeat called tachycardia, as well as an abnormally slow heartbeat, known as bradycardia. Extrasystoles are premature contractions of the entire heart or of its individual components. Ventricular fibrillation or cardiac fibrillation describes irregular activity of the ventricles characterized by complete failure of the heart's pumping capacity and over 350 contractions per minute.

For therapy of acute bradycardial cardiac arrhythmias and for preoperative, intraoperative and postoperative cardiac stimulation, the user can choose between various modes of operation and set the associated parameters via an input device. Examples of possible modes of operation include:

1. Asynchronous mode of operation: In asynchronous mode of operation, ventricular or atrial stimulating pulses are delivered with a fixed base frequency regardless of the heart's natural rhythm. In this mode of operation, the frequency is input as a parameter.

2. Inhibited mode of operation: In the absence of spontaneous cardiac activity, ventricular and atrial stimulating pulses are delivered to the heart with a base frequency set as a parameter.

3. Asynchronous dual chamber mode of operation: Regardless of the heart's natural rhythm, the atrium and ventricle are stimulated with the base frequency set as a parameter. The stimulus in the ventricle is delivered after the atrial stimulus, by a delay equal to the atrioventricular conduction time which has been set.

4. Dual chamber mode of operation without atrial stimulation: In this mode of operation, which is used in the absence of spontaneous cardiac activity, ventricular stimulating pulses are delivered to the heart with a base frequency set as a parameter. In this mode of operation, sensing is possible in both channels.

5. Dual chamber universal mode of operation: In this mode of operation, stimulation and sensing are possible in both channels. If spontaneous cardiac activity is not detected, the atrium and ventricle are stimulated with a base frequency assigned as a parameter. In this case the stimulus in the ventricle is delivered after the atrial stimulus, by a delay equal to the assigned atrioventricular conduction time.

Any desired combination of the cited modes of operation is also possible. The frequency, atrial sensitivity, atrial stimulation amplitude, atrioventricular conduction time, ventricular stimulation amplitude, ventricular sensitivity, and the post ventricular refractory time can be assigned as parameters for the various modes of operation. The mode of operation currently selected as well as the current values of the parameters is shown in the display device. In existing cardiac stimulation devices, a liquid crystal display is usually provided as the display device. Selection of the mode of operation and assignment of the associated parameters is performed via keys and/or rotary knobs, which are placed on the housing of the unit next to the display device. A disadvantage in this respect is that it takes time to become familiar with correct input of the mode of operation and associated parameters, since the correlation of the keys and rotary knobs to the respective modes of operation and associated parameters is not clear enough to be immediately obvious to the user. Consequently, not only is the input process relatively complicated, but also increases the risk of entering inappropriate modes of operation or parameters if not operated properly, thus causing a health risk for the patients.

The Invention and Its Advantages

In contrast to this, the cardiac stimulation device described as the invention with the features of claim 1 has the advantage that there is not a long learning curve for the user to be able to select the mode of operation and set the associated parameters that are required for the patient. For this purpose a touch screen is provided as a combined input and display device. The user selects the mode of operation by touching the screen with a finger or a stylus. As soon as the mode of operation has been selected, only those parameters that must be entered for the selected mode of operation are displayed on the touch screen. For this purpose, the touch screen can be subdivided into different areas. In doing so, it is particularly advantageous to display the mode of operation in the upper area of the touch screen, while the associated parameters are displayed in the lower areas. Touching a parameter displayed on the screen activates input of the parameter in question. For input of values, increment and decrement keypads, incremental encoders or a keypad with numerals 0 to 9 can be provided. In the case of an increment and decrement keypad, the value of the parameter is increased by actuating the increment key and decreased by actuating the decrement key. The keypads either can be integrated in the touch screen or provided beside the touch screen in the housing. By combining input and display devices on the one hand and the display of the parameters associated with the selected mode of operation on the other hand, input is limited to the data required at that time. By subdividing the touch screen into different areas for mode of operation and the associated parameters, the presentation is clearly organized. Thus not only can the mode of operation and parameters be entered more quickly and simply than in existing devices, but also operation is more reliable than in the case of input via keys and rotary knobs. The danger of entering modes of operation or parameters that are inappropriate for the patient is reduced compared to existing devices, as is the associated health risk.

In order to adapt the parameters displayed on the touch screen to the mode of operation currently selected, the processor of the device is programmed with appropriate intelligent software, which is capable of context-sensitive interpretation of the entries made by the user and context-sensitive control of the layout of the touch screen.

According to an advantageous configuration of the invention, an emergency input device is provided in addition to the combination input and display device. This makes it possible to be able to react as quickly as possible in an emergency. When the emergency input device is actuated, an emergency mode of operation is selected, in which, for example, the heart is stimulated with a base frequency of 80 pulses per minute, an amplitude of 12 volts and a pulse length of 0.75 ms.

Furthermore, an input device for starting high-rate stimulation, otherwise known as overdrive pacing, can be provided. If atrial tachycardia is detected, the user can switch to the high-rate mode of operation by actuating the input device. In this mode of operation the stimulation frequency is increased to 400 pulses per minute without input of further parameters. Furthermore, the previously selected mode of operation is not deactivated. By continuous actuation of the input device, for example by pressing a high-rate select key, the user can start asynchronous overstimulation, for example. If the input device and the decrement key are actuated simultaneously, the stimulation frequency during high-rate stimulation is automatically and continuously lowered to the previously assigned natural frequency of the heart, such as 70 pulses per minutes. In most cases, tachycardia will be ended in this way. If actuation of the high-rate select key is ended, the device reverts to the previously assigned mode of operation. In this way it is especially easy and fast to start and end high-rate stimulation.

According to a further advantageous configuration of the invention, a locking input device is provided for locking and unlocking the combined input and display device. To ensure that no input can take place if the combined input and display device is accidentally or inadvertently touched, thereby changing the mode of operation and/or the assigned parameters, the input and display device is locked once input is complete. In the locked state, touching the input and display device will not cause any input. If the mode of operation and/or the assigned parameters are to be changed, the input and display device must first be unlocked. For this purpose, a key located separately from the input and display device can be provided.

Further advantages and advantageous configurations of the invention will become apparent from the description hereinafter and from the diagram and claims.

### DIAGRAM

An example of the design of the invention is illustrated in the diagram, wherein:

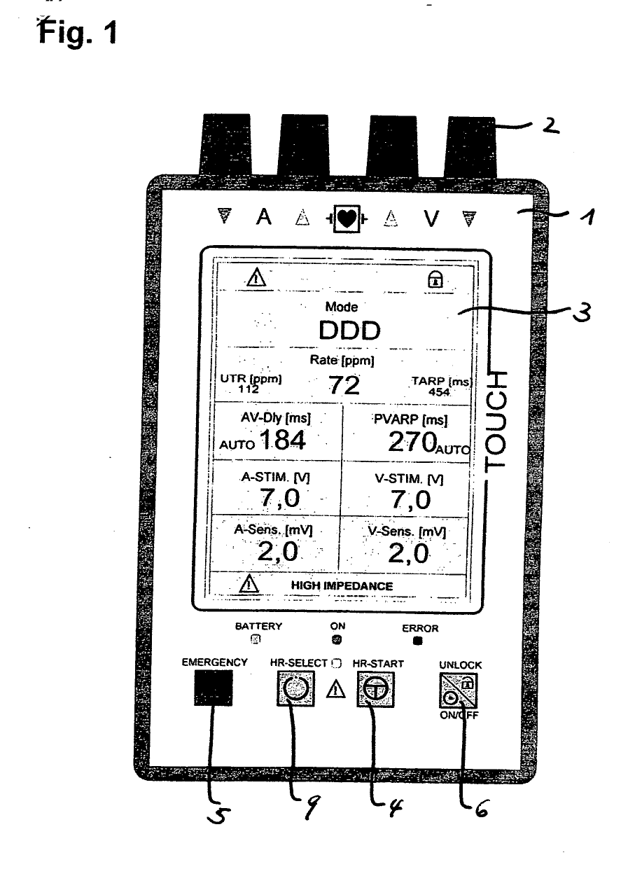

FIG. 1 shows a front view of a first design example of a cardiac stimulation device,

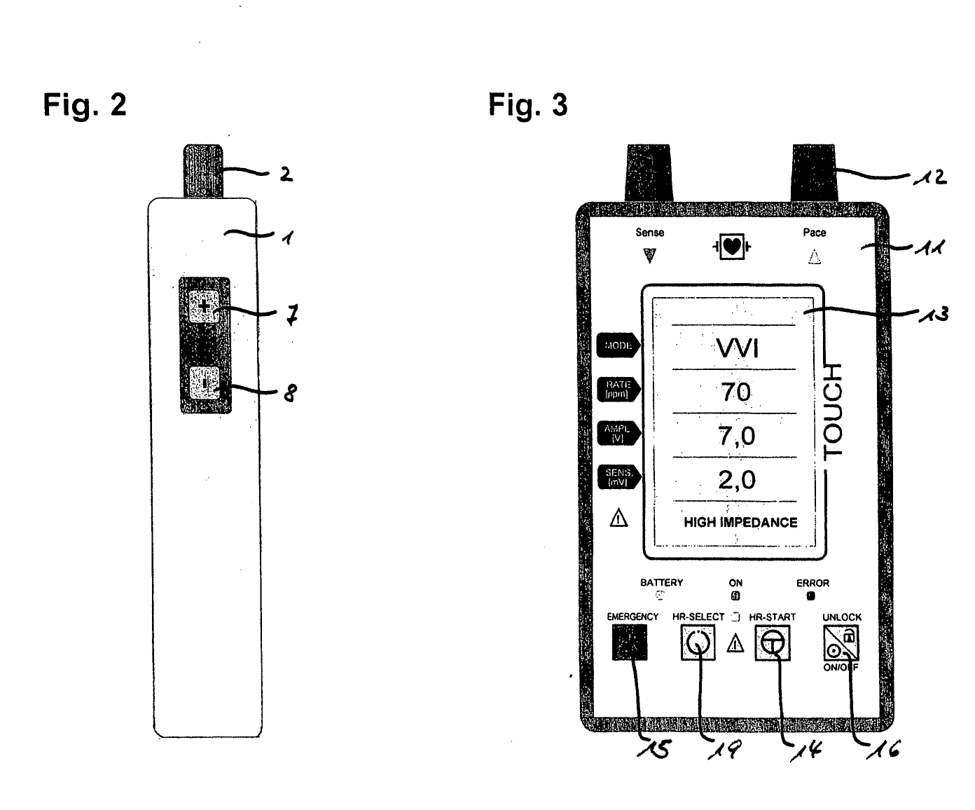

FIG. 2 shows a side view of the cardiac stimulation device according to FIG. 1,

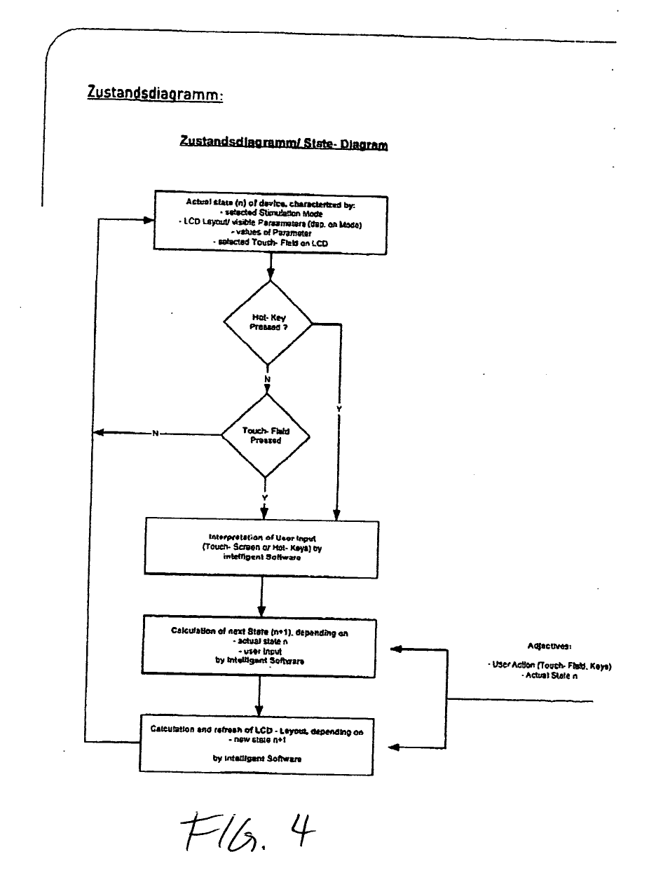

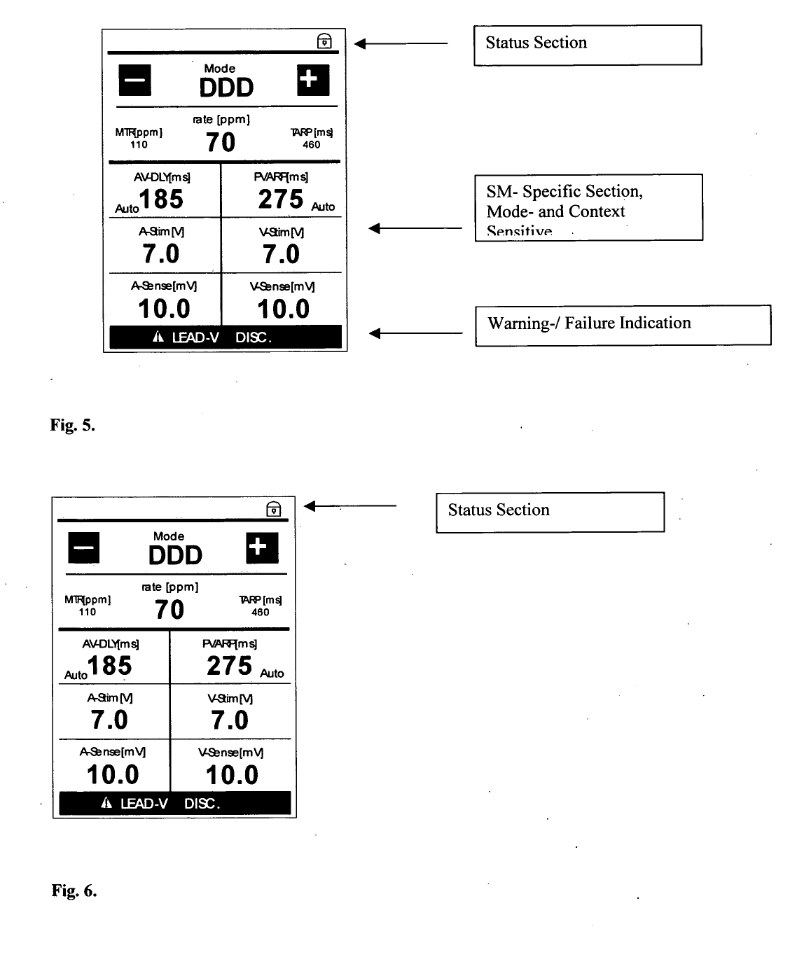

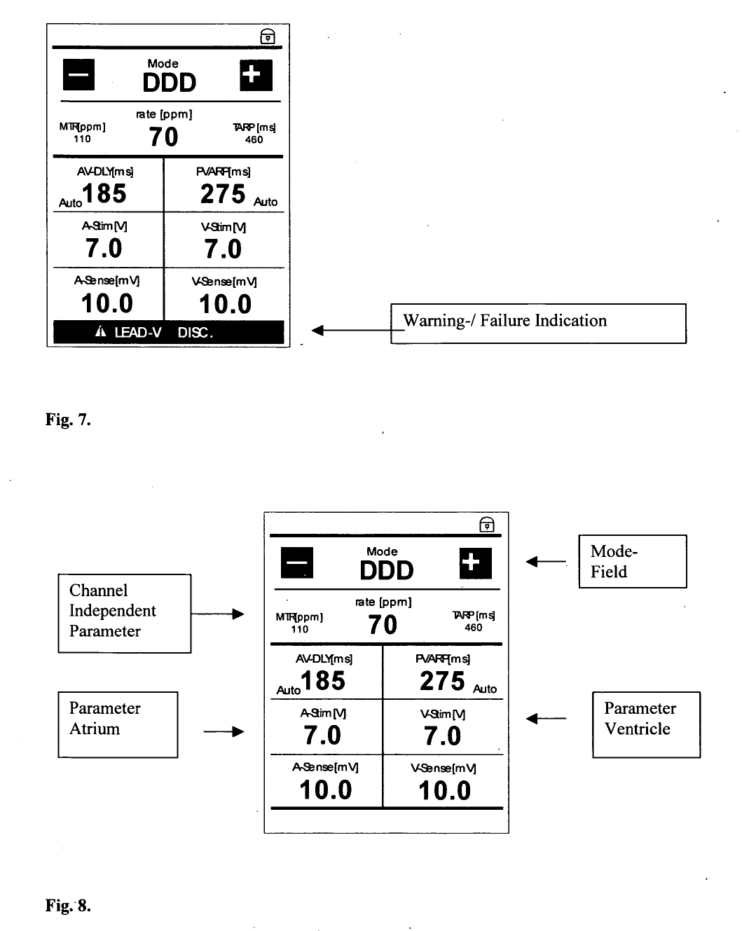

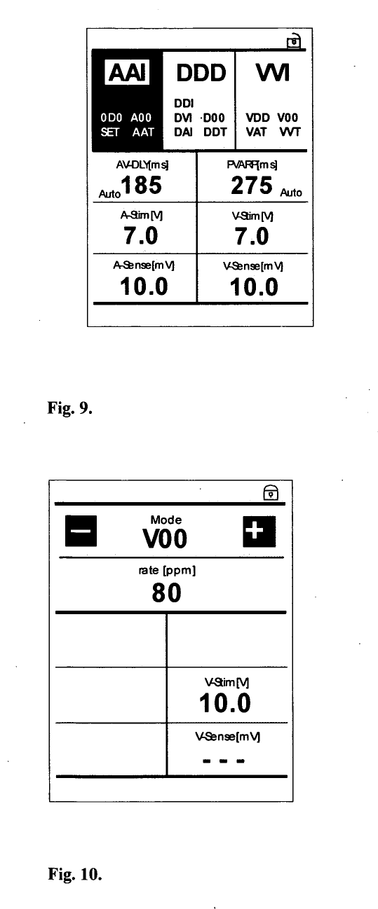

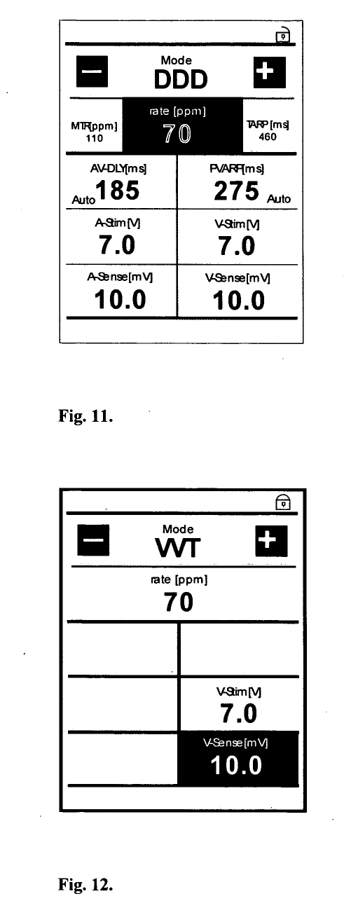

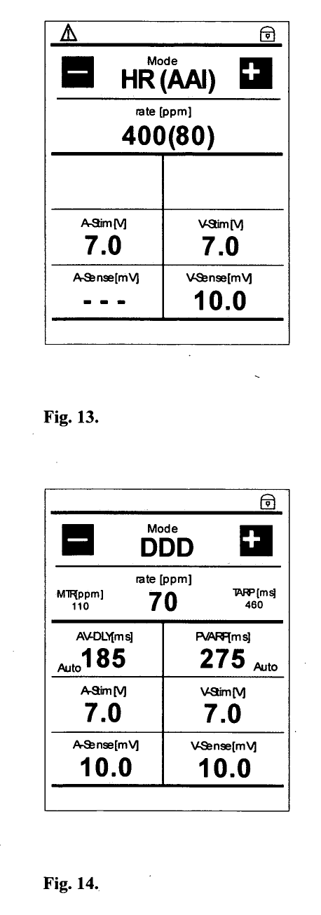

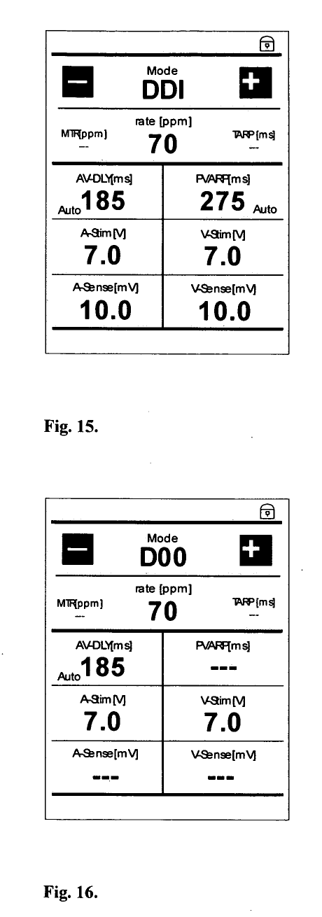

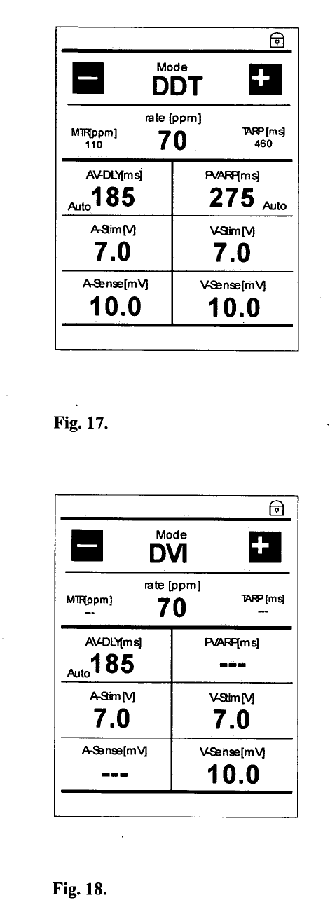

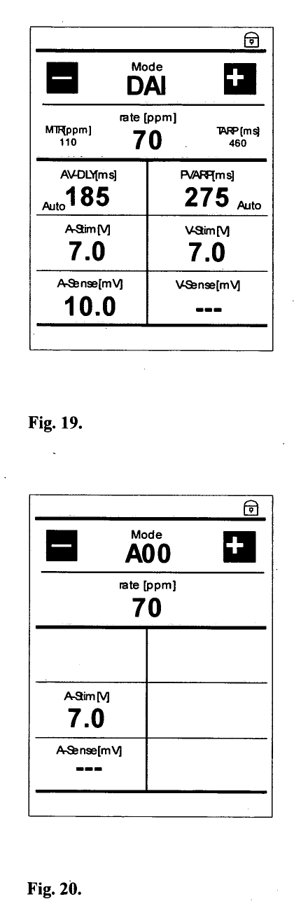

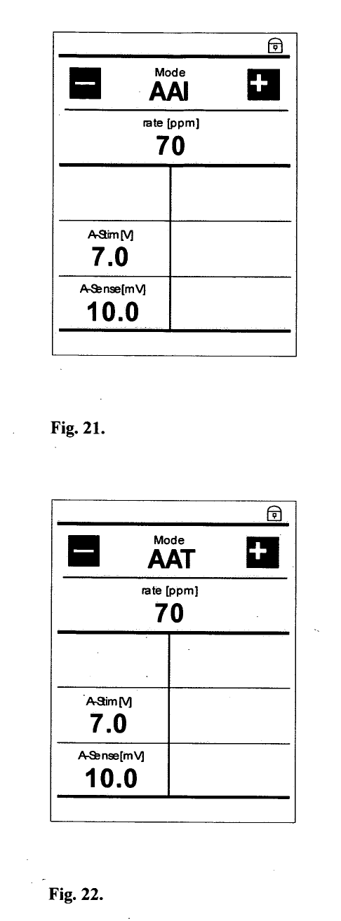

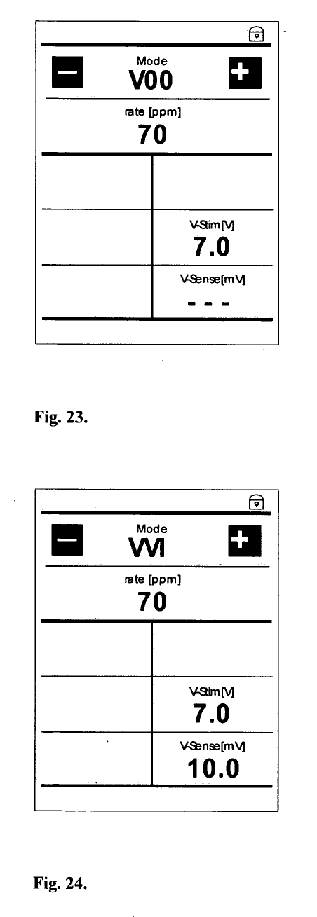

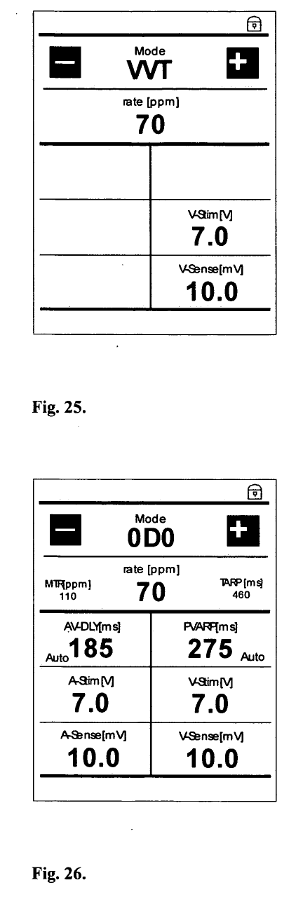

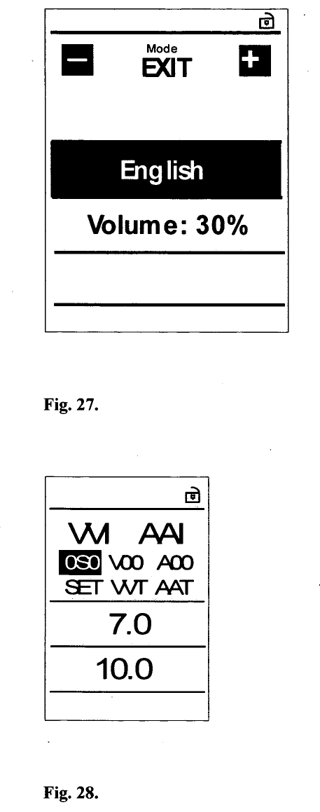

FIG. 3 shows a front view of a second design example of a cardiac stimulation device,

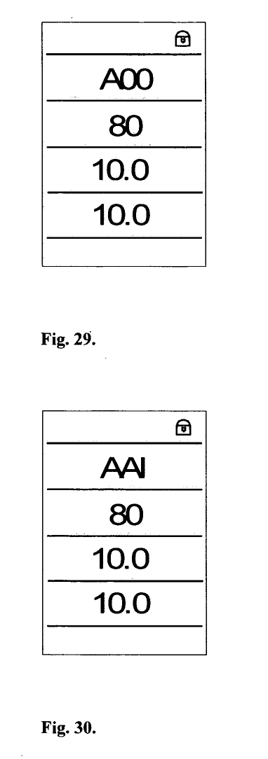

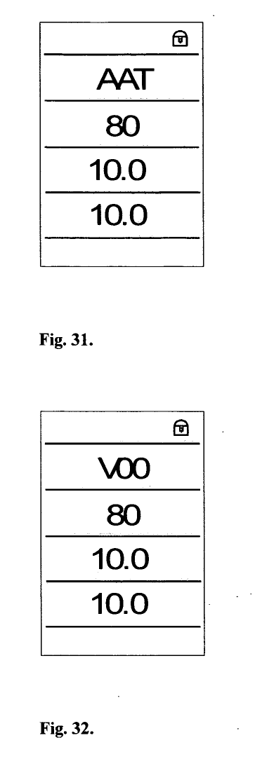

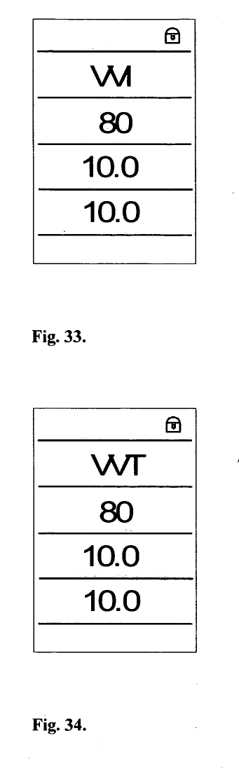

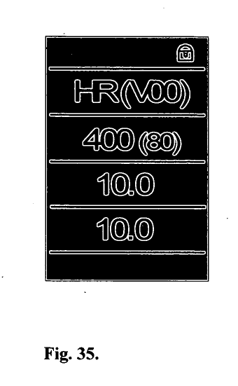

FIG. 4 shows a state diagram of the devices according to FIGS. 1, 2, and 3.

### DEVICE FOR CARDIAL STIMULATION WITH TOUCH SCREEN

Description of the Design Example

FIG. 1 shows a cardiac stimulation device with a housing 1, a total of four leads 2 for stimulation electrodes, which are not illustrated in the drawing, a combined input and display device 3 designed as a touch screen, a key 5 as an emergency input device and a key 6 as a locking input device. FIG. 2 shows the device according to FIG. 1 in side view. Increment and decrement keys 7 and 8 are visible in this illustration.

The device is used for atrial and ventricular stimulation. It is therefore also known as a dual chamber device.

On the touch screen evident in FIG. 1, the dual chamber universal mode of operation DDD is selected as the mode of operation in the uppermost section. The parameters illustrated in the fields below it relate to the selected mode of operation. To change mode of operation, the device is unlocked by actuation of locking input device 6. The mode of operation and/or an associated parameter is then changed by touching the associated field on the touch screen of input and display device 3 with the finger or a stylus. If the desired field is activated, the user can change the mode of operation or the parameter by actuation of increment and decrement keys 7 and 8. After completion of the input process, the device is locked once again by actuation of the locking input device. Locking takes place automatically if no input is made within a predetermined time interval. A high-rate select key 9, shown as “HR Select” in FIG. 1, is used to select the high-rate stimulation mode of operation. Key 4 is used to start high-rate stimulation.

FIG. 3 illustrates a second design example of a cardiac stimulation device. In contrast to the design example according to FIGS. 1 and 2, this is a device for stimulation of only one chamber. For this reason only two leads 12 are provided for stimulation electrodes. Combined input and display device 13 provided in housing 11 is subdivided into fewer areas than the input and display device 3 of the device according to FIGS. 1 and 2. A touch screen is also involved. With regard to key 14 for starting high-rate stimulation, key 15 of the emergency input device, key 16 of the locking input device, high-rate select key 19, as well as the increment and decrement keys, which are not visible in FIG. 3, the two devices are identical.

FIG. 4 shows a state diagram for the devices according to FIGS. 1, 2 and 3. The device is in state n, which is characterized by the mode of operation and the parameters that have been selected. The mode of operation and parameters are illustrated on the display. A query is displayed as to whether the key of the emergency input device has been pressed. If this is the case, the user's input is interpreted by the processor and the associated software. If the key of the emergency input device has not been pressed, a check is run to determine whether instructions are being input via the combined input and display device. If this is not the case, the device remains in state n. On the other hand, if data are being input by the user via the input and display device, an interpretation of the input takes place. On the basis of the mode of operation and/or of the associated parameters entered by the user, the state n+1 is calculated by the processor via the software. The mode of operation and the parameters of state n+1 are then displayed on the combined input and display device.

All advantages and advantageous configurations of the invention can be integral to the invention, both individually and in any combination with one another.

List of Reference Numerals

1 Housing

2 Leads

3 Combined input and output device

4 Key for starting high-rate stimulation

5 Key of the emergency input device

6 Key of the locking input device

7 Increment key

8 Decrement key

9 High-rate select key

10

11 Housing

12 Leads

13 Combined input and output device

14 Key for starting high-rate stimulation

15 Key of the emergency input device

16 Key of the locking input device

17

18

19 High-rate select key

Appartaus Method and Software for Presentation of User Interface on the Touchscreen

1. Main Topics:

Hardware for the implementation of a universal, temporary (not implanted), externally portable two chamber pacemaker.

Method of operation.

Design of the context sensitive menu structure of the display and the user interface by which the system is operated:
no nested menus, one menu level only.
implementing the parameters quickly, safely and intuitively.
resulting in a short learning time.

2. Implementation:

Portable, handheld unit, containing:
Display
Processor
Functional interface to cardiac leads

Operation via touch screen and hot keys on the front panel increment/decrement keys on the edge

Software for the system operation and the implementation of the user interface, Real time operating system, task oriented

Secured plug sockets for connecting the leads to the implanted electrodes

3. Operational Requirements, User Interface:

User interface is menu operated with characteristics as follows:
Context sensitive display organisation, only mode relevant parameters are displayed
No nested menus, one level only.

Liquid crystal display with touch screen operation
All stimulation relevant parameters can be verified at first glance
system status indication
failure-/warning indication

Hotkeys for emergency operation
High rate stimulation of Atrium Tachycardias/fibrillation/flutter
Emergency stimulation (VOO/70 ppm 10V/1 ms)

Increment/decrement keys on the edge

FIG. 1

[image]

4. Implementation/Embodiment:

4.1 Sectioning of the display: LCD-area is partitioned into 3 Sections, which can be processed and read out independently of each other:

upper section: status line, indication of system status.

middle section: operation and indication of pacemaker specific parameters

bottom section: indication/output of warnings, failure indications and critical system status.

[image]

Implementation by means of intelligent software using a real time operating system

Individual tasks are processed separately.
=>Task for the input of keyboard operation by the user
=>Task for the input of the touch-screen
=>Task for the context sensitive interpretation of the inputs via keyboard and touch-screen
=>Task for the context sensitive output on the display
=>Task for the evaluation and indication of system status
=>Task for the evaluation and indication of warnings and failures/failure modes
=>Task for the stimulation in accordance with the selected stimulation mode/stimulation program

Tasks interact with each other via messages and common domains of variables.

4.2 Section Status Line: Indication of system status

[image]

Symbols:

| | |
|---|---|
| [symbol] | Warning Symbol: user to mind status line and warning/failure |
| | indications and to check the operating manual |
| [symbol] | Lock-Symbol: keyboard locked. No entries possible. |
| [symbol] | Unlock-Symbol: keyboard unlocked. Entries masked, no entries |
| | possible. |
| [symbol] | Electrode Inputs Open |
| [symbol] | Electrode Inputs short circuit |
| [symbol] | Battery-Warning EOL-1 |
| [symbol] | Battery-Warning EOL-2 |
| [symbol] | Battery-Warning “Empty” or “Not Equipped” |
| [symbol] | Backup-Battery EOL- Warning |
| [symbol] | Backup-Battery Empty |
| [symbol] | Rate above 150 ppm |

4.3 Section Status Line: Indication of system status

[image]

Warnings- and Failure Indications

Attention: Every Warning-/Failure Indication via Warning Symbol

LOW IMP. A Low Impedance Atrium (<=200 Ohm)

LEAD-A SHORT. Short Circuit of Electrodes Atrium

LOW IMP. V Low Impedance Ventricle (<=200 Ohm)

LEAD-V SHORT. Short Circuit of Electrodes Ventricle

HIGH IMP. A High Impedance Atrium (>1000 Ohm)

LEAD-A DISC. Electrodes Contact Ventricle open (high impedance)

HIGH IMP. V High Impedance Ventricle (>1000 Ohm)

LEAD-V DISC. Electrodes Contact Ventricle open (high impedance)

NOISE A Noise Atrium

NOISE V Noise Ventricle

Battery EOL 1: EOL-1 Warning Battery

Battery EOL 2: EOL-2 Warning Battery

Battery Empty: Battery Empty

No Battery: No Battery or Operation via Backup- Battery

Back.-Batt: Low Low Voltage Backup-Battery

Back.-Batt: Empty Backup-Battery Empty

ST CPU Failure Self Test CPU

ST RAM Failure Self Test RAM

ST ROM Failure Self Test ROM (Program code)

ST. STIM. A Failure Self Test Stimulation Amplifier Atrium

ST. STIM. V Fairlure Self Test Stimulation Amplifier Ventricle

ST. SENSE. A Failure Self Test Sense Amplifier Atrium

ST. SENSE. V Failure Self Test Sense Amplifier Ventricle

4.4 Pacemaker specific section of LCD: mode and context sensitive

For Mode Selection and for Adjustment of Mode Specific Parameters

[image]

Display Area Partitioning in:
Parameters valid for Atrium or for Actions started in Atrium: left Side: A-Sense, A-Stim, AV-DLY
Parameters valid for Ventricle or for Actions started in Ventricle: right Side: V-Sense, V-Stim, PVARP
Channel Independent Parameters: RATE, MTR, TARP
Superordinated Mode-Field

Selection of modes via touch-screen (touch-fields) after selection of mode-touch field either directly via touch-screen or via +/− keys on the edge.

[image]

After Mode Selection the display structure is adapted context sensitively according to the selected mode as follows:
Only parameters relevant to the respective simulation mode are displayed.
Parameters not required for the respective mode are masked out.
Parameters not required for the respective mode however being of interest are indicated with “- - - ” and can be activated and edited temporarily.
All indicated parameters can be verified at first glance. one menu level only.
In Atria Stim.-Modes (Voo, VVI, VVT) only relevant Atria parameters are indicated, in Ventricular Stim.-Modes (V00, VVI, VVT) only relevant Ventricular parameters.

Implementation of the Context Sensitive Menu Structure of the display in accordance with the Flow Diagram in the Appendix.

Example: Mode V00:
Atria Parameter s are masked out
V-Sense not relevant but interesting and therefore editable

[image]

Menus of all Modes see Appendix

Selection and Adjustment of Mode Sensitive Parameters also via Touch-Screen (Touch-Fields) as well as via +/− Keys on the Edge and/or +/− Keys on Touch-Screen.

Possible only when Keyboard/Touch unlocked

Example Rate Adjustment in DDD-Mode

[image]

Example: Sensitivity Adjustment in Mode VVT

[image]

Implementation of the Context Sensitive Menu Structure of the Display in Accordance with the Flow Diagram in the Appendix.

4.5 Hot Keys for Emergency Operation

For Emergency Operation two Hot Keys are provided, enabling immediate, fast reaction of the user

HR-Stimulation:
Activation of high-rate-stimulation via keys HR-SELECT and HR-START in the unlocked state.
HR-stimulation active only as long as HR-Start-keys suppressed. p1 Before and after HR-stimulation background stimulation in the last selected SM-mode is performed.
HR-stimulation with automatic “Slow-Down” as described in the earlier German Patent Application.
Context sensitive menu structure of the display for HR-stimulation as follows.
In ( ) SM-Mode before HR-Select with the rate valid for this Mode

[image]

Emergency Stimulation:
Activation of the Emergency Stimulation via Key EMERGENCY in the unlocked State
Emergency-Stimulation: V00/70 ppm/10v/1 ms. Parameters are adjusted automatically
Context Sensitive Menu Structure of the LCD as follows:

[image]

4.6 Overview of menu structures of all modes

Mode-Selection via touch and/or +/− keys on the edge:

[image]

[image]

[image]

[image]

[image]

[image]

[image]

[image]

[image]

[image]

1. A cardiac stimulation device
with leads (2, 12) for at least two stimulating electrodes,
with an input device for input of an operational mode suitable for the treatment and for selection of parameters,
with a display device for displaying the selected mode of operation and parameters,
with a processor for defining electrical stimulating pulses from the selected mode of operation and parameters,
with a stimulation device connected to the leads (2, 12) for generating the electrical stimulating pulses defined by the processor,
characterized in that
a combined input and display device (3, 13), which is designed as a touch screen for entering data and instructions is provided.

2. A control unit according to claim 1, characterized in that increment and decrement keypads (7, 8) are provided as an additional input device for changing the value of a parameter.

3. A control instrument according to claim 1, characterized in that an additional emergency input device (5, 15, 9, 19) for entering a mode of operation and the associated parameters appropriate for an emergency is provided.

4. A control instrument according to claim 1, characterized in that a locking input device (6, 16) for locking and unlocking the combined input and display equipment is provided.

5. A control unit according to claim 1, characterized in that the touch screen is subdivided into multiple areas, in that one area is provided for display and input of the mode of operation, and in that the other areas are provided for displaying and entering various parameters.
